(19) United States
(12) Patent Application Publication
(10) Pub. No.: US 2026/0174670 A1
(43) Pub. Date: Jun. 25, 2026

(54) FLAVOR-DEPLETED, FUNCTIONALIZED CITRUS FIBER

(71) Applicant: Herbstreith & Fox GmbH & Co. KG Pektin-Fabriken, Neuenbürg (DE)

(72) Inventors: Gerhard F. FOX, Neuenbürg (DE); Matthias ROTH, Neuenbürg (DE)

(21) Appl. No.: 18/719,590

(22) PCT Filed: Dec. 14, 2022
(86) PCT No.: PCT/EP2022/085874

§ 371 Date: Jun. 13, 2024

(30) Foreign Application Priority Data
Dec. 15, 2021 (DE) 10 2021 133 198.6

(51) Int. Cl. A61K 9/00; A61K 36/84

(52) U.S. Cl. CPC A61K 9/0014; A61K 36/84; A61K 2300/00

(57) ABSTRACT

The present invention relates to a flavor-depleted, functionalized citrus fiber and to a method for the production thereof. The invention also relates to the use of the flavor-depleted, functionalized citrus fiber as a thickening or structuring agent in various industrial products. The invention further relates to a food product, a feed product, a food supplement, a beverage, a cosmetic product, a pharmaceutical product, a detergent product, cleaning product or medical product, which has been produced using a flavor-depleted, functionalized citrus fiber.

The present invention relates to a flavoring-depleted, functionalized citrus fiber and to a method for the production thereof. The invention also relates to the use of the flavoring-depleted, functionalized citrus fiber as a thickening or structuring agent in various industrial products. Furthermore, the invention relates to a mixture of the flavoring-depleted, functionalized citrus fiber with an isolated pectin. Finally, the invention relates to a food product, feed product, food supplement, beverage, cosmetic product, pharmaceutical product, detergent product, cleaning product or medical product which has been produced using the above-mentioned flavoring-depleted, functionalized citrus fiber.

### BACKGROUND TO THE INVENTION

Dietary fibers are largely indigestible food components, mostly carbohydrates, which are predominantly found in plant-based foods. For the sake of simplicity, dietary fibers are divided into water-soluble fibers such as pectin and water-insoluble fibers such as cellulose. Dietary fiber is considered an important component of human nutrition.

The consumption of dietary fiber is considered to be beneficial to health. The water-soluble fiber in food increases the volume of food without significantly increasing the energy content. If they are not already sufficiently swollen before ingestion, they absorb more water in the stomach. The resulting increase in volume leads to an increase in the feeling of satiety. Dietary fiber also prolongs the time that the food remains in the intestine or stomach. Water-soluble fibers such as pectin bind bile acids of the cholesterol metabolism in the intestine and thus lead to a lowering of the cholesterol level.

Soluble dietary fiber in particular is said to reduce glucose absorption, slow down glucose adsorption and starch processing and control postprandial glucose levels in the serum. People who consume a lot of dietary fiber have a reduced risk of numerous lifestyle diseases, in particular obesity, high blood pressure, coronary heart disease (CHD), stroke, diabetes and various gastrointestinal diseases. Accordingly, the German Nutrition Society (DGE) recommends a daily intake of at least 30 g of dietary fiber.

The use of citrus fibers as dietary fibers in the production of food is becoming Increasingly important. One reason for this is the fact that citrus fibers are a mixture of insoluble dietary fibers such as cellulose and soluble dietary fibers such as pectin and thus ideally provide the health-promoting spectrum of effects listed above. By using citrus fibers, the functional properties of food products can be specifically optimized and adjusted, for example in terms of viscosity, emulsion formation, gel formation, dimensional stability or texture. Citrus fibers can thus replace other less accepted or even harmful additives in foods and, as non-e-classified substances, lead to simpler product labeling and thus to increased product acceptance.

Citrus fibers, which have a limited range of applications, are usually obtained using the processes known in the prior art. This is because they either have a relatively high content of primary and possibly secondary flavoring substances- and therefore regularly have a relatively intense aroma of their own- or unsatisfactory functional properties.

Volatile organic compounds (VOCs), which can be perceived via the nose or palate using olfactory receptors and which, individually or in mixtures, give food in particular a desired smell or taste, are referred to as flavorings or aroma-active substances. In the food sector, a distinction is made between primary flavorings, which occur in the intact cell of fruit, for example, and secondary flavorings, which are formed from—usually non-volatile—precursors through enzymatic, oxidative or thermal conversion. Flavors, which give food its sweet, sour, salty or bitter taste, are not considered to be flavorings. A large number of flavors can be traced back to volatile aroma-active substances that belong to the class of aromatics, e.g. furans and alkyl pyrazines, carboxylic acids, esters, terpenes, aldehydes or ketones.

The use of previously known citrus fibers, which have a relatively high content of primary and possibly secondary flavoring substances, can lead to sensory impairment in the end product. Such a sensory impairment can, for example, result in the fruit flavor of a fruit-containing food product only being able to develop to a limited extent or being masked. On the other and, unprocessed citrus fibers that have been stored for a longer period of time of the aforementioned type and/or the products manufactured using such citrus fibers regularly exhibit off-flavors, for example as a result of exposure to air and/or oxygen and chemical reactions triggered thereby. The term off-flavor here means any odor or taste or mouthfeel that is not normally associated with the respective end product. For example, the smell of R-carvone (smell of spearmint, Mentha spicata) can be perceived, particularly after prolonged storage of the aforementioned citrus fibers, as a result of the oxidation of R-limonene (orange-like smell) in air.

Citrus fibers, which have a lower content of flavorings and therefore do not have the aforementioned disadvantages or have them to a lesser extent, are usually produced in the prior art using solvents, which impair or deteriorate the functional properties of the fibers. The solvents are in particular alkaline solutions.

There is therefore a need for citrus fibers with the widest possible range of applications and for new processes to produce them.

The present invention is based on the object of improving the prior art or offering an alternative to it.

### SUMMARY OF THE INVENTION

According to a first aspect of the present invention, the object is achieved by means of a method for producing a flavoring-depleted, functionalized citrus fiber, wherein the method comprises the following steps:
(a) providing a raw material containing cell wall material of an edible citrus fruit;
(b) digestion of the raw material, which includes
   i. incubation of an aqueous suspension of the raw material at an acidic pH value or
   ii. enzymatic treatment of an aqueous suspension of the raw material and/or
   iii. introduction of mechanical energy into an aqueous suspension of the raw material;
(c) single or multi-stage separation of the digested and flavoring-depleted material of step (b) from the aqueous liquid containing a first fraction of flavorings;
(d) extraction of a second fraction of flavorings by contacting the material separated in step (c) with a water-based extraction medium at a temperature of 30° C. to 90° C.;
(e) separation of the flavoring-depleted material of step (d) from the water-based extraction medium;
(f) extraction of a third fraction of flavorings by contacting the material separated in step (e) with an alcohol-based extraction medium;
(g) separation of the flavoring-depleted material of step (f) from the alcohol-based extraction medium;
(h) drying the material separated in step (g) to obtain the flavoring-depleted, functionalized citrus fiber,
wherein steps (d) and (e) are optionally carried out and the separation in step (g) is carried out such that the separated flavoring-depleted material has a dry matter content of ≥30% by weight, preferably ≥35% by weight, particularly preferably ≥40% by weight and especially preferably ≥45% by weight.

The production method according to the invention results in citrus fibers with a large inner surface area, which also increases the water-binding capacity and is accompanied by good viscosity formation.

These fibers are functionalized fibers which have a satisfactory strength due to the production method according to the invention. To obtain the optimum rheological properties such as viscosity, gelling or texturing, the user may need to apply additional shear forces.

As the inventors have found, the functionalized citrus fibers produced by the method according to the invention have good rheological properties, which can be characterized, for example, by their water-binding capacity. The fibers according to the invention can be easily rehydrated and the advantageous rheological properties are retained even after rehydration.

The production method according to the invention results in citrus fibers which-due to the depletion of aroma-active substances or flavorings—are highly neutral in taste and odor and are therefore advantageous for use in the food sector. The inherent flavor of the other ingredients is not masked and can therefore develop optimally. The citrus fibers obtained by means of the method described here not only have a very low proportion of aroma-active substances-compared to previously known citrus fibers ˜ and thus an essentially neutral inherent aroma. They also advantageously have functional properties which can be varied within a relatively wide range of values and can thus be adapted for a large number of applications. Consequently, the method claimed here can be used to produce citrus fibers which have a wide range of applications.

The citrus fibers according to the invention are obtained from citrus fruits and thus represent natural ingredients with known positive properties,

Plant processing residues such as citrus pomace or citrus pulp can be used as a raw material in the production method according to the invention. These processing residues are inexpensive, are available in sufficient quantities and offer a sustainable and ecologically sensible source for the citrus fibers according to the invention.

Citrus fibers are established and accepted in the food industry, so that corresponding compositions can be used immediately and internationally without lengthy approval procedures.

### The Invention in Detail

Citrus fruits and preferably processing residues of citrus fruits can be used as raw material. Accordingly, citrus peel- and here preferably citrus albedo and/or citrus flavedo, citrus vesicles, segment membranes, i.e. citrus membranes, or a combination thereof can be used as the raw material for use in the method according to the invention. In a preferred manner, citrus pomace is used as the raw material, i.e. the pressing residues of citrus fruits, which typically also contain the fruit flesh in addition to the peel, or citrus pulp, i.e. the residues of the juice-producing industry, which contain the pectin and cellulose material of the inner, juice-containing part of the respective citrus fruit.

The citrus flavedo has glands which contain essential oils, also known as essential peel oils. Therefore, in step (a) of the method described herein, a raw material is particularly preferably provided in which essential oils contained in the Citrus flavedo have been at least partially removed under the action of mechanical energy, advantageously by means of a puncture of the Citrus flavedo, in particular of glands arranged in the Citrus flavedo and containing essential oils, or by removing the glands containing essential oils. Alternatively or additionally, providing the raw material in step (a) may comprise removing the essential oils contained in the Citrus flavedo. For example, a puncture of the citrus flavedo, in particular of glands arranged in the citrus flavedo and containing essential oils, may be provided or a removal of the glands containing essential oils. During and/or after such a puncture, the essential oil is preferably supplied to a water bath, from which it may be extracted for further use.

All citrus fruits known to the person skilled in the art can be used as citrus fruits. By way of example and not limitation, the following are listed here: Mandarin (Citrus reticulata), Clementine (Citrus×aurantium Clementine group, Syn.: Citrus Clementina), Satsuma (Citrus×aurantium Satsuma group, Syn. Citrus Unshiu), Mangshan (Citrus Mangshanensis), Orange (Citrus×aurantium orange group, syn.: Citrus sinensis), Bitter orange (Citrus×aurantium bitter orange group), Bergamot (Citrus×limon bergamot group, syn. Citrus bergamia), grapefruit (Citrus maxima), grapefruit (Citrus\hat{ }aurantium grapefruit group, syn.: Citrus paradisi) pomelo (Citrus×aurantium pomelo group), true lime (Citrus×aurantiifolia), common lime (Citrus×aurantiifolia, syn. Citrus lati folia), kaffir lime (Citrus hystrix), Rangpur lime (Citrus×jambhiri), lemon (Citrus×limon lemon group), citron (Citrus medica) and kumquats (Citrus japonica, syn.: Fortunella). Preferred are the orange (Citrus×aurantium orange group, syn.: Citrus sinensis) and the lemon (Citrus×limon lemon group).

According to the present invention, the digestion of the raw material in step (b) of the method comprises
incubation of an aqueous suspension of the raw material at an acidic pH value or
enzymatic treatment of an aqueous suspension of the raw material and/or
introduction of mechanical energy into an aqueous suspension of the raw material.

In one embodiment of the method, chemical digestion is provided by incubating an aqueous suspension of the raw material at an acidic pH value.

The chemical digestion in step (b) of the method serves to remove pectin by converting the protopectin into soluble pectin and simultaneously activating the fiber by increasing the inner surface area. Furthermore, the raw material is thermally comminuted by the digestion. In other words, the acidic incubation in an aqueous environment under the influence of heat causes the pectin to dissolve and consequently the cell structure to disintegrate. The raw material breaks down into citrus fibers. This results in thermal comminution, which means that a mechanical comminution step is not absolutely necessary as part of the production method. This represents a decisive advantage over conventional fiber production processes, which in contrast require a shearing step, such as (high) pressure homogenization, in order to obtain a fiber with sufficient rheological properties, as can be defined on the basis of the water binding capacity. It is particularly advantageous that the chemical digestion in step (b) and the associated thermal comminution can also release and separate encapsulated flavorings from the raw material.

The raw material is present as an aqueous suspension during chemical digestion. According to the invention, a suspension is a heterogeneous mixture of substances composed of a liquid and finely dispersed solids (raw material particles).

To obtain the aqueous suspension, the liquid used is accordingly an aqueous liquid. According to the invention, an aqueous liquid is defined as a liquid comprising more than 50% by volume, preferably more than 60%, 70%, 80% or even 90% by volume of water. In a preferred embodiment, the aqueous solution contains no organic solvent and in particular no alcohol.

The aqueous liquid can be, for example, deionized water, tap water or an aqueous salt solution with an ionic strength of I<0.2 mol/L.

As the suspension tends towards sedimentation and phase separation, the particles are suitably kept in suspension by shaking and/or stirring. There is therefore no dispersion in which the particles are crushed by mechanical action (shearing) so that they are finely dispersed.

To achieve an acidic pH value, the skilled person can use any known acid or acidic buffer solution. For example, an organic acid such as citric acid can be used.

Alternatively or in combination with this, a mineral acid can also be used. Examples include sulphuric acid, hydrochloric acid, nitric acid or sulphurous acid. Nitric acid is preferred.

In the chemical digestion in step (b) of the method, the pH value of the suspension is between pH=0.5 and pH=4.0, preferably between pH=1.0 and pH=3.5 and particularly preferably between pH=1.5 and pH=3.0.

During chemical digestion, incubation takes place at a temperature between 60° C. and 95° C., preferably between 70° C. and 90° C. and particularly preferably between 75° C. and 85° C. For example, the chemical digestion can be carried out at a temperature of 70° C., 71° C., 72° C., 73° C., 74° C., 75° C., 76° C., 77° C., 78° C., 79 C, 80° C., 81° C., 82° C., 83° C., 84° C., 85° C., 86° C., 87° C., 88° C., 89° C. or 90° C.

As the inventors have discovered, these high incubation temperatures cause a significant extraction of low-molecular organic flavorings such as aldehydes, esters, carboxylic acids and terpenes, even without the presence of organic solvents.

The efficient digestion and the resulting comminution of the fiber material also considerably releases encapsulated flavorings, which are thus accessible for extraction.

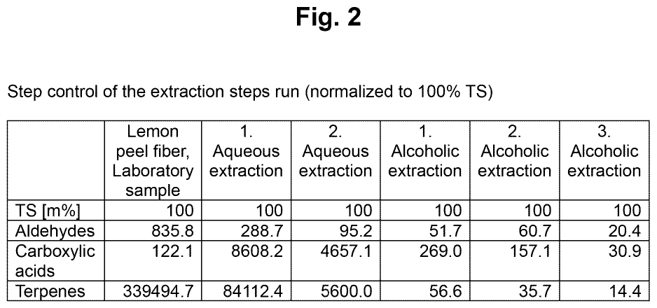

Incubation in an acidic environment has the additional advantage that the carboxylate anions contained in the raw material are protonated and the resulting uncharged carboxylic acids can be extracted more easily. The inventors were also able to prove this by means of an organic chemical analysis accompanying the method, which showed a strong increase in the carboxylic acid concentration after acid hydrolysis (see table in FIG. 2).

Incubation takes place over a period of between 60 minutes and 8 hours and preferably between 2 hours and 6 hours.

During chemical digestion, the aqueous suspension suitably has a dry mass of between 0.5% and 5% by weight, preferably between 1% and 4% by weight, and particularly preferably between 1.5% and 3% by weight.

The aqueous suspension is stirred and/or shaken during chemical digestion. This is preferably carried out continuously so that the particles are kept suspended in the suspension.

As an alternative to chemical digestion or acid hydrolysis, biochemical digestion may be provided in step (b) of the method, namely by enzymatic treatment of an aqueous suspension of the raw material.

The biochemical digestion in step (b) of the method also serves to remove pectin by converting the protopectin into soluble pectin and simultaneously activating the fiber by increasing the inner surface area. In this case, the raw material is also comminuted by the digestion, but in a milder way, because the enzymatic treatment is preferably carried out without supplying thermal energy, in particular at room temperature. In other words, the enzymatic treatment in an aqueous environment causes the pectin to be dissolved out and, as a result, the cell structure to disintegrate. The raw material breaks down into citrus fibers. This results in enzymatic comminution in particular, meaning that a mechanical comminution step is not absolutely necessary as part of the production method. This represents a decisive advantage over conventional fiber production processes, which in contrast require a shearing step, such as (high) pressure homogenization, in order to obtain a fiber with sufficient rheological properties. It is particularly advantageous that the biochemical digestion in step (b) and the associated enzymatic comminution in particular can also release and separate encapsulated flavorings from the raw material.

As the inventors have discovered, biochemical digestion without the presence of organic solvents also results in a significant extraction of low-molecular organic flavorings such as aldehydes, esters, carboxylic acids and terpenes.

Due to the efficient biochemical digestion and the resulting comminution of the fiber material, encapsulated flavorings are also released, which are thus accessible for extraction.

The raw material is also present as an aqueous suspension during biochemical digestion. A definition of the term aqueous suspension is given above. As the suspension tends towards sedimentation and phase separation, the particles are suitably kept in suspension by shaking and/or stirring. There is therefore no dispersion here either, in which the particles are crushed by mechanical action (shearing) so that they are finely dispersed.

The biochemical digestion in step (b) is advantageously carried out with one or more of the following enzymes: pectinases (EC 3.2.1.15, EC 4.2.2.10), cellulases (EC 3.2.1.4, EC 3.2.1.74, EC 3.2.1.91) or hemicellulases, where hemicellulases are enzymes which catalyze the biochemical degradation of hemicellulose. Selected examples of hemicellulases are endo-1,4-beta-xylanase (EC 3.2.1.8), endo-1,4-mannanase (EC 3.2.1.78), endo-1,3-beta-glucanase (EC 3.2.1.39), exo-1,4-beta-xylosidase (EC 3.2.1.37), exo-1,3-beta glucanase (EC 3.2.1.58) and exo-1,4-beta-mannobiohydrolase (EC 3.2.1.100).

Alternatively or in addition to a chemical or biochemical digestion in step (b) of the method, a mechanical digestion may be provided, namely by introducing mechanical energy into an aqueous suspension of the raw material. A definition of the term aqueous suspension is given above.

The introduction of mechanical energy into the aqueous suspension of the raw material in step (b) is advantageously carried out using a process selected from the group consisting of high shear treatment, pressure homogenization, colloidal grinding, extrusion, ultrasonic treatment, microfluidizer, spider disperser and combinations thereof.

During mechanical digestion, the raw material is initially present as an aqueous suspension, in particular the swollen raw material. By applying mechanical energy, preferably without applying thermal energy, particularly at room temperature, the swollen raw material particles are broken down so that they are finely dispersed. A dispersion is thus produced. Mechanical comminution alone or in combination with chemical or biochemical digestion can also advantageously release and separate encapsulated flavoring substances from the raw material.

If in step (b) of the claimed method only mechanical digestion is carried out, fibers with sufficient rheological properties are also obtained as a result of the associated mechanical comminution step.

If in step (b) a chemical digestion by means of an acid hydrolysis described above and a mechanical digestion or a biochemical digestion and a mechanical digestion are carried out, the sequence of the digestions provided in each case can be freely selected. Advantageously, chemical digestion in the form of acid hydrolysis or biochemical digestion is carried out in a first step and mechanical digestion in a second step. The latter, in particular, carries out a further comminution step.

The mechanical comminution of the fiber material also considerably releases encapsulated flavorings, which are thus accessible for extraction.

In step (c) of the method, the digested and flavoring-depleted material of step (b) is separated from the aqueous liquid, which contains a first fraction of hydrophilic, i.e. water soluble, flavorings, and thus recovered. This separation is carried out as a single-stage or multi-stage separation. The first fraction of flavorings already contains a large number of flavorings, for example from the classes of aldehydes, carboxylic acids, esters and terpenes. It is primarily the carboxylic acids that can be separated particularly efficiently in this first extraction step, as the heavy extractable carboxylates present in the starting material are profaned by the chemical digestion in step (b) and can only be extracted from the material by washing as a result. Alternatively, the carboxylates are also profanated by adjusting the pH value in the strongly acidic pH range at the end of the biochemical digestion and thus converted into extractable carboxylic acids.

The separation in step (c) is advantageously carried out with a decanter, a separator and/or a belt press, preferably with a decanter and/or separator.

In an advantageous manner, the digested material is subjected to a multi-stage separation. Here it is preferable if the separation from the aqueous liquid is carried out in stages with the separation of increasingly finer particles. This means, for example, that in a two-stage separation, both stages separate larger particles, wherein finer particles are separated in the second stage compared to the first stage in order to achieve the most complete possible separation of the particles from the aqueous liquid. Preferably, the first separation of particles is carried out with decanters and the second separation with separators. As a result, the material becomes more and more finely particulate with each separation step. Alternatively or additionally, a belt press can also be used.

After the chemical (in particular acidic) or biochemical digestion, possibly in combination with a mechanical digestion, in step (b) and the separation of the digested material, an optional step (d) may be provided in which the material separated in step (c) is brought into contact with a water-based extraction medium, at a temperature of 30° C. to 90° C. This step results in the extraction of a second fraction of hydrophilic, i.e. water-soluble, flavoring substances. In addition, water-soluble substances remaining in the material separated in step (c), such as sugar, can be removed. The removal of sugar in particular contributes to the citrus fiber being less adhesive and therefore easier to process and use.

In the context of the invention, the “water-based extraction medium” is understood to be an aqueous liquid which consists of more than 50% by volume, preferably more than 60, 70, 80 or even 90% by volume of water. In a preferred embodiment, the aqueous solution contains no organic solvent and in particular no alcohol.

The water-based extraction medium can be, for example, deionized water, tap water or an aqueous salt solution with an ionic strength of I<0.2 mol/1.

The mixture of this water-based extraction medium and the material separated in the previous step is referred to as an “aqueous extraction mixture”.

The water-based extraction medium advantageously has a pH value of 1 to 7, preferably 2 to 6 and particularly preferably 3 to 5.

The extraction of the second fraction of flavorings according to step (d) is advantageously carried out at a temperature between 30° C. and 90° C., preferably between 40° C. and 80° C. and particularly preferably between 50° C. and 70° C.

Contact with the water-based extraction medium advantageously takes place over a period of between 10 minutes and 2 hours, preferably between 30 minutes and 1 hour.

In the extraction according to step (d), the dry mass in the aqueous extraction mixture consisting of the water-based extraction medium and the material separated in step (c) is advantageously between 0.1% by weight and 5% by weight, preferably between 0.5% by weight and 3% by weight and particularly preferably between 1% by weight and 2% by weight.

It may be provided that the extraction according to step (d) is carried out in a batch process.

More advantageously, the extraction according to step (d) is carried out with mechanical agitation of the aqueous extraction mixture. This is more expediently carried out by stirring and/or shaking the aqueous extraction mixture.

By means of the extraction according to step (d), a further depletion of hydrophilic, i.e. water-soluble, flavoring substances in the raw material takes place.

According to one method variant, coarse or undigested particles are separated from the extraction mixture consisting of the water-based extraction medium and the separated material by wet sieving.

As the inventors have found, the acidic or biochemical digestion of the raw material in aqueous suspension and/or the introduction of mechanical energy into the aqueous raw material suspension followed by bringing the suspension into contact with a water-based extraction medium already results in a significant extraction of low molecular weight organic flavoring substances such as aldehydes, esters, carboxylic acids and terpenes, even without the presence of organic solvents.

If extraction step (d) is carried out using the water-based extraction medium, this is followed by step (e), in which the material of step (d), which is further depleted in flavorings, is separated from the water-based extraction medium. This separation is advantageously carried out with the aid of a decanter, a belt press and/or a separator, preferably with the aid of a decanter and/or separator.

According to one variant of the method, the extraction according to step (d) with subsequent separation according to step (e) is carried out several times.

In step (f), a further extraction step is then carried out, but using an alcohol-based extraction medium. A third fraction of flavoring substances is extracted, which contains flavoring substances that are less hydrophilic or hydrophobic than the flavoring substances contained in the first and second fractions. For differentiation purposes, the flavoring substances contained in the first and second fractions are referred to as hydrophilic, and the flavoring substances contained in the third fraction are referred to as hydrophobic.

The mixture of the alcohol-based extraction medium and the material separated in the previous step is referred to as the “alcoholic extraction mixture”.

The alcohol-based extraction medium is advantageously an alcohol which may be selected from the group consisting of methanol, ethanol and isopropanol.

A further variant of the method provides that the alcohol-based extraction medium advantageously has a proportion of alcohol of between 50 and 95% by volume, preferably of between 55 and 80% by volume, and particularly preferably of between 60 and 70% by volume. The proportion of alcohol in the alcohol-based extraction medium may, for example, be 51, 53, 55, 57, 69, 61, 63, 65, 67, 69, 71, 73, 75, 77, 79, 81, 83, 85, 87, 89, 91 or 93% by volume.

The extraction step is advantageously carried out at a temperature between 40° C. and 75° C., preferably between 50° C. and 70° C. and particularly preferably between 60° C. and 65° C.

Contact with the alcohol-based extraction medium advantageously takes place over a period of between 60 min and 10 h and preferably between 2 h and 8 h.

During extraction with the alcohol-based extraction medium, the dry mass in the alcoholic extraction mixture consisting of the alcohol-based extraction medium and the material separated in the previous step is advantageously between 0.5% and 15% by weight, preferably between 1.0% and 10% by weight, and particularly preferably between 1.5% and 5.0% by weight.

The extraction with the alcohol-based extraction medium can be carried out with mechanical agitation, in particular stirring and/or shaking, of the alcoholic extraction mixture, advantageously in a container with an agitator.

One embodiment of the method provides for the method to be carried out discontinuously or continuously. In the case of a discontinuous process, the alcohol-based extraction medium is treated with an aroma adsorbing cleaning medium, in particular activated carbon, after separation from the flavoring-depleted material. The alcohol-based extraction medium is advantageously stirred and/or shaken in the presence of activated carbon in a first step and isolated by decantation, centrifugation or filtration in a second step. Alternatively or additionally, rectification can be provided.

In one embodiment of the method, if the digestion in step (b) comprises or consists of an acid hydrolysis, a partial neutralization is carried out during and/or after extraction with the alcohol-based extraction medium by adding at least one sodium or potassium salt, preferably NaOH or KOH.

During and/or after extraction with the alcohol-based extraction medium, the material can also be decolorized. This decolorization can be carried out by adding one or more oxidizing agents. Examples include the oxidizing agents chlorine dioxide and hydrogen peroxide, which can be used alone or in combination.

Each extraction step with the alcohol-based extraction medium comprises contacting the flavoring-depleted material with the alcohol-based extraction medium for a certain period of time, followed by separating the hydrophilic and hydrophobic flavoring-depleted material from the alcohol-based extraction medium according to step (g), wherein the separation is carried out such that the separated flavoring-depleted material has a dry matter content of ≥30% by weight, preferably ≥35% by weight, particularly preferably ≥40% by weight and especially preferably ≥45% by weight. The separation step (g) thus also includes a desolventization step, which results in a material that has a high dry substance content and a correspondingly low content of extraction medium. The desolventization achieved in step (g) by the separation methods used is based on a physical separation of solid and solvent (e.g. squeezing, pressing, decanting or centrifuging), in contrast with chemical desolventization, which is achieved, for example, by distilling off the extraction medium. Such chemical desolventization has the disadvantage that the flavorings present in the extraction medium remain in the solid material and therefore no reduction of the flavorings takes place.

As a result of the separation step (g) according to the invention, far smaller quantities of aroma-active substances present in this extraction medium are carried over into subsequent method steps (such as drying). It has been shown that this significantly improves the depletion of flavorings from the citrus fiber.

In one variant of the method, the aforementioned separation is carried out with the aid of a screw press, a decanter, a belt press and/or a separator, preferably with the aid of a screw press.

According to one embodiment of the method, the extraction according to step (f) with subsequent separation according to step (g) is carried out several times, in particular twice or three times, the separation being carried out at least in the last of the several extraction steps in such a way that the flavoring-depleted material separated off in the last step has a dry substance content of ≥30% by weight, preferably ≥35% by weight, particularly preferably ≥40% by weight and especially preferably ≥45% by weight.

To carry out the extraction twice according to step (f) with subsequent separation according to step (g) in each case, the separation can therefore be carried out while maintaining a dry substance content of ≥30% by weight, during the first and second separation or only during the second separation, wherein it is preferred that the separation is carried out while maintaining a dry substance content of ≥30% by weight only in the second, I.e. the last separation step according to step (g).

For a threefold execution of the extraction according to step (1) with subsequent separation according to step (g) in each case, the separation can thus be carried out while maintaining a dry substance content of ≥30% by weight, in the first, second and third separation, or in the second and third separation, or only in the third separation, wherein it is preferred that the separation is carried out while maintaining a dry substance content of ≥30% by weight only in the third, i.e. the last separation step according to step (g).

According to an advantageous alternative or supplementary embodiment, extraction with the alcohol-based extraction medium is carried out in a countercurrent process. This is particularly advantageous in the case of a multi-stage extraction using the alcohol-based extraction medium. The separation of the material depleted in hydrophilic and hydrophobic flavorings takes place continuously during the extraction process when using the countercurrent method. In this procedure, the separation step (g) can be an integral part of the extraction step (f), but it can also be carried out in the conventional manner as a downstream separation step. This simplifies and accelerates the process and makes it less error-prone.

According to an advantageous embodiment, the final concentration of alcohol in the alcoholic extraction mixture increases with each extraction step during at least two extractions with an alcohol-based extraction medium. This incrementally increasing proportion of alcohol reduces the water content in the fiber material in a controlled manner, so that the rheological properties of the fibers are maintained during the subsequent solvent extraction and drying steps and no collapse of the functionalized fiber structure occurs.

A further variant of the method provides that the final concentration of the alcohol in the first extraction step is between 50% by volume and 95% by volume, preferably between 60% by volume and 70% by volume, in the second extraction step between 60% by volume and 95% by volume, preferably between 70% by volume and 85% by volume, and in an optional third extraction step between 70% by volume and 95% by volume, preferably between 80% by volume and 90% by volume.

Before drying in step (h), an additional step can be provided by means of which the alcohol is partially or completely removed by bringing the material depleted of hydrophilic and hydrophobic flavoring substances into contact with water vapor. A stripper is preferably used for this purpose, in which the material depleted of flavoring substances is brought into contact with water vapor as stripping gas in countercurrent flow. This further step can also be included in step (g) or step (h).

In step (h), the material separated in step (g) and depleted of hydrophilic and hydrophobic flavoring substances is dried, followed by the stripped material, if applicable, wherein the drying comprises drying under normal pressure or by means of vacuum drying.

Examples of suitable drying processes using normal pressure are fluidized bed drying, fluidized bed drying, belt drying, drum drying or paddle drying. Fluidized bed drying is particularly preferred here. This has the advantage that the product is dried in a loosened state, which simplifies the subsequent grinding step. In addition, this type of drying avoids damage to the product due to local overheating thanks to the easily controlled heat input.

In an alternative embodiment, the drying according to step (h) comprises vacuum drying and preferably consists of vacuum drying. During vacuum drying, the separated material depleted of hydrophilic and hydrophobic flavorings and subsequently, if applicable, stripped material is exposed to a vacuum as dry material, which reduces the boiling point and thus leads to evaporation of the water and any alcohol still contained, even at low temperatures. The heat of vaporization continuously extracted from the dry material is suitably supplied from outside until the temperature remains constant. Vacuum drying has the effect of lowering the equilibrium vapor pressure, which favors capillary transport. This has proven to be particularly advantageous for the citrus fiber material in question, as it preserves the activated, opened fiber structures and thus the resulting rheological properties. Preferably, vacuum drying takes place at a vacuum of less than 400 mbar, preferably less than 300 mbar, more preferably less than 250 mbar and particularly preferably less than 200 mbar.

According to an advantageous embodiment, the method additionally comprises a comminution, grinding and/or sieving step after drying in step (h). This is advantageously configured such that, as a result, at least 90% by weight of the particles have a particle size of less than 250 μm, preferably a particle size of less than 200 μm and in particular a particle size of less than 150 μm. At this particle size, the fiber is easily dispersible and has an optimum swelling capacity.

In a second aspect, the invention provides a flavoring-depleted, functionalized citrus fiber obtainable by or obtained by the production method described herein.

In a third aspect, the invention provides a functionalized flavoring-depleted citrus fiber, which has a total content of aroma-active aldehydes of less than 500 μg/kg toluene equivalents, advantageously of less than 100 μg/kg toluene equivalents, preferably of less than 50 μg/kg toluene equivalents, particularly preferably of less than 10 μg/kg toluene equivalents. Preferably, said citrus fiber is obtainable by the method described herein or is obtained thereby.

In a fourth aspect, the invention provides a functionalized, flavoring-depleted citrus fiber, which has a total content of aroma-active carboxylic acids of less than 500 μg/kg toluene equivalents, advantageously of less than 100 μg/kg toluene equivalents, preferably of less than 50 μg/kg toluene equivalents, particularly preferably of less than 10 μg/kg toluene equivalents. Preferably, said citrus fiber is obtainable by the method described herein or is obtained thereby.

In a fifth aspect, the invention provides a functionalized flavoring-depleted citrus fiber which has a total content of aroma-active terpenes of less than 1000 μg/kg toluene equivalents, advantageously of less than 100 μg/kg toluene equivalents, preferably of less than 10 μg/kg toluene equivalents, more preferably of less than 1 μg/kg toluene equivalents. Preferably, said citrus fiber is obtainable by the method described herein or is obtained thereby.

In a sixth aspect, the invention provides a functionalized flavoring-depleted citrus fiber, which has a water-binding capacity of more than 5 g/g, preferably of more than 10 g/g, particularly preferably of more than 15 g/g and especially preferably of more than 20 g/g. The water-binding capacity can, for example, be more than 5 g/g, 6 g/g, 7 g/g, 8 g/g, 9 g/g, 10 g/g, 11 g/g, 12 g/g, 13 g/g, 14 g/g, 15 g/g, 16 g/g, 17 g/g, 18 g/g, 19 g/g, 20 g/g, 21 g/g, 22 g/g, 23 g/g, 24 g/g or 25 g/g.

According to the invention, a functionalized flavoring-depleted citrus fiber is provided which has a total content of aroma-active esters of less than 500 μg/kg of toluene equivalents, advantageously of less than 100 μg/kg of toluene equivalents, preferably of less than 50 μg/kg of toluene equivalents, particularly preferably of less than 10 μg/kg of toluene equivalents. Preferably, said citrus fiber is obtainable by the method described herein or is obtained thereby.

For the flavoring-depleted functionalized citrus fiber, the features of aspects three to six described above may also be used in any desired combination. Thus, in a particular embodiment, the citrus fiber claimed herein may have all of the features of aspects three through six described, wherein the citrus fiber preferably can be or is obtained by the method claimed herein.

Advantageously, the functionalized flavoring-depleted citrus fiber has a dietary fiber content of 80% to 95% by weight.

In a seventh aspect, the invention relates to the use of a flavoring-depleted functionalized citrus fiber according to one of the embodiments described further above as a thickening agent or structuring agent in a food product, a feed product, a beverage, a dietary supplement, a cosmetic product, a pharmaceutical product, a detergent product, a cleaning agent product or a medical product.

In an eighth aspect, the invention relates to a mixture comprising a flavoring-depleted functionalized citrus fiber according to one of the embodiments described further above and an isolated pectin, which is preferably a low esterified pectin, a high esterified pectin or a low esterified amidated pectin, or a mixture thereof.

In a ninth aspect, the invention relates to a food product, feed product, dietary supplement, beverage, cosmetic product, pharmaceutical product, detergent product, cleaning agent product or medical device produced using a flavoring-depleted functionalized citrus fiber according to one of the embodiments further described above.

### Definitions

A citrus fiber according to the application is a component consisting mainly of fibers Isolated from a non-lignified vegetable cell wall of a citrus fruit and consisting mainly of cellulose. The term fiber is in some respects a misnomer because the citrus fibers do not appear macroscopically as fibers, but are a powdery product. Other components of citrus fiber include hemicellulose and pectin.

A pectin according to the application is defined as a plant polysaccharide which, as a polyuronide, essentially consists of a-1,4-glycosidically linked D-galacturonic acid units. The galacturonic acid units are partially esterified with methanol. The degree of esterification describes the proportion of carboxyl groups in the galacturonic acid units of the pectin, which are present in esterified form, e.g. as methyl esters.

According to the invention, a highly esterified pectin is understood to be a pectin which has a degree of esterification of at least 50%. A low-esterified pectin, on the other hand, has a degree of esterification of less than 50%. The degree of esterification describes the percentage of carboxyl groups in the galacturonic acid units of the pectin which are present in esterified form, e.g. as methyl esters. The degree of esterification can be determined using the method according to JECFA (Monograph 19-2016, Joint FAO/WHO Expert Committee on Food Additives).

According to the present invention, flavoring agents or aroma-active substances are defined in sensory terms as those volatile organic compounds which, individually or in a mixture, impart a desired odor or taste, in particular to foods, i.e. are aroma-relevant. This includes both primary flavoring substances, which occur, for example, in fruits in the intact cell, and secondary flavoring substances, which are formed from—mostly non-volatile—precursors by enzymatic, oxidative or thermal conversions. Flavorings, which give foods a sweet, sour, salty or bitter taste, are not considered flavorings. A large number of flavors are due to volatile aroma-active substances that belong to the class of aromatics, e.g. furans and alkyl pyrazines, carboxylic acids, esters, terpenes, aldehydes or ketones.

“Flavorings” or “aroma-active substances” according to the present inventions are defined in chemical terms as those volatile organic compounds which can be detected by needle trap GC/MS and belong to the classes of aromatics, e.g. furans and alkylpyrazines, carboxylic acids, esters, terpenes, aldehydes or ketones. The group of aroma-active aldehydes includes the following compounds, wherein the list is not to be understood as limiting: Acetaldehyde, propanal, butanal, (E)-2-butanal, pentanal, (E)-2-pentenal, hexanal, (E)-2-hexanal, heptanal, (E)-2-heptenal, octanal, (E)-2-octenal, nonanal, (E)-2-nonenal, decanal, (E)-2-decenal, 2-methylpropanal, 2-methyl-2-propanal, 3-methylbutanal, 2-methylbutanal, benzaldehyde. The group of aroma-active carboxylic acids includes the following compounds, wherein the list is not to be understood as being exhaustive: formic acid, acetic acid, propionic acid, pentanoic acid, hexanoic acid. The group of aroma-active terpenes includes the following compounds, wherein the list is not to be understood as being exhaustive: alpha-pinene, limonene, p-cymene, valencene.

Volatile organic compounds according to the present invention are carbon-containing substances which have a vapor pressure of 0.01 kilo Pascal or more at 293.15 Kelvin or have a corresponding volatility under the respective conditions of use.

In a further aspect, “flavoring agents” or “flavor-active substances” according to the present invention are definable by the sensory properties, as far as they are defined as such volatile organic compounds which, individually or in a mixture, impart a desired odor or taste in particular to foods, i.e. are flavor-relevant. This includes both primary flavoring substances, which occur, for example, in fruits in the intact cell, as well as secondary flavoring substances, which are formed from—mostly non-volatile—precursors by enzymatic, oxidative or thermal conversions. Flavors, which give foods a sweet, sour, salty or bitter taste, are not considered flavorings, A large number of flavors are due to volatile aroma-active substances that belong to the class of aromatics, e.g. furans and alkyl pyrazines, carboxylic acids, esters, terpenes, aldehydes or ketones.

According to the present invention, terpenes are a group of chemical compounds with a variety of carbon skeletons, the common feature of which is that their basic skeleton can be traced back to isoprene units. This includes both pure hydrocarbons and thus terpenes in the narrower sense as well as the group of terpenoids. Terpenoids are characterized by the fact that their basic structure can also be derived from isoprene units, wherein the carbon structure can also be partially modified and/or comprise additional functional groups. A non-limiting list of functional groups includes alcohols, ethers, aldehydes, ketones, carboxylic acids, carboxylic acid esters and glycosides.

At this point, it should be explicitly pointed out that features of the solutions described above or in the claims and/or figures can also be combined, if necessary, in order to be able to implement or achieve the features, effects and advantages explained in a correspondingly cumulative manner.

All the features disclosed in the application documents are claimed as being essential to the invention, provided that they are new, either individually or in combination with one another, compared with the prior art.

It should also be expressly pointed out that in the context of the present patent application, indefinite articles and numerical indications such as “one”, “two” etc. are: generally to be understood as “at least” indications, i.e. as “at least one . . . ”, “at least two . . . ” etc., unless it is expressly apparent from the respective context or it is obvious or technically mandatory for the person skilled in the art that only “exactly one . . . ”, “exactly two . . . ” etc. can be meant there.

Further advantages, special features and useful further embodiments of the invention can be seen from the sub-claims and the following illustration of preferred embodiments with reference to the figures.

### EXEMPLARY EMBODIMENTS

The embodiments shown here are only examples of the present invention and should therefore not be understood to be limiting. Alternative embodiments contemplated by the skilled person are equally encompassed by the scope of protection of the present invention.

### 1. Description of the Production Method Based on a Rough Flow Diagram

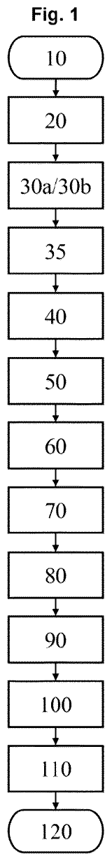

FIG. 1 shows a schematic flow diagram of a method according to the invention for producing the flavoring-depleted, functionalized citrus fiber. Starting from the citrus pomace 10, the pomace is digested by a digestion 20, for example incubation in an acidic solution at 70° to 80° C. by hydrolysis and with the addition of mechanical energy. This is followed by two separate steps 30a (decanter) and 30b (separator) to separate as completely as possible all particles of the digested flavoring-depleted material from the aqueous liquid containing the first fraction of flavorings. The separated material is subjected to a water-based extraction 35. Here, the separated material is brought into contact with a water-based extraction medium at a temperature of 40° C. to 80° C. for a period of 30 minutes to 1 hour. Coarse or undigested particles are separated from the aqueous extraction mixture consisting of the water-based extraction medium and the separated material by wet sieving. In step 40, the separation of the flavoring-depleted material from the water-based extraction medium containing the second fraction of flavorings. A total of three alcohol-based extraction steps are then carried out. Two of these extraction steps (50 and 70) are carried out in a countercurrent process with subsequent solid-liquid separation by means of decanters 60 and 80, wherein the extraction of the third and fourth fractions of flavorings takes place. The third extraction step 90 is also carried out in a countercurrent process with subsequent solid-liquid separation using a screw press (100) in order to obtain a dry substance content of ≥45% by weight and a fifth fraction of flavorings. Finally, in step 110, the fibers are vacuum dried to obtain the citrus fibers 120 according to the invention.

### 2. Test Method for Determining the Grain Size

### Measuring Principle:

In a sieving machine, a set of sieves is arranged one above the other, with the mesh size increasing from the bottom sieve to the top. The sample is placed on the top sieve—the one with the largest mesh size. The sample particles with a larger diameter than the mesh size remain on the sieve; the finer particles fall through to the next sieve. The proportion of the sample on the different sieves is balanced and expressed as a percentage.

### Implementation:

The sample is weighed to two decimal places. The sieves are fitted with sieving aids and stacked on top of each other with increasing mesh size. The sample is quantitatively transferred to the top sieve, the sieves are clamped and the sieving process runs according to defined parameters. The individual sieves are weighed with sample and sieving aid as well as empty with sieving aid. If only one limit value in the particle size spectrum is to be checked for a product (e.g. 90% by weight <250 μm), then only one sieve with the corresponding mesh size is used.

Measurement specifications:
Sample quantity: 15 g
Sieving aids: 2 per sieve tray
Screening machine: AS 200 digit, Retsch GmbH
Screen movement: three-dimensional
Vibration height: 1.5 mm
Sieving duration: 15 min

The screen structure consists of the following mesh sizes in pm: 1400, 1180, 1000, 710, 500, 355, 250, 200, 150 followed by the bottom.

The grain size is calculated using the following formula:

$$Proportion per sieve in \% = \frac{weight in g on the sieve \times 100}{sample weight in g}$$

### 3. Test Method for Determining the Water-Binding Capacity

### Implementation:

The sample is left to swell with excess water for 24 hours at room temperature. After centrifugation and subsequent decanting of the supernatant, the water binding capacity can be determined gravimetrically in g H₂O/g sample. The pH value in the suspension must be measured and documented.

The following parameters must be observed:

### Sample Weight:

plant fiber: 1.0 g (in centrifuge tube)
water addition: 60 ml
centrifugation: 4000×g
centrifugation time 10 min

20 minutes after the start of centrifugation (or 10 minutes after the end of centrifugation), separation of the supernatant water from the swollen sample. The sample with the bound water is balanced.

The water binding capacity (WBV) in g H₂O/g sample can now be calculated using the following formula:

$$WBV ( g {H}_{2} O / g sample ) = \frac{sample with bound water ( g ) - 1. g}{1. g}$$

### 4. Test Method for Determining the Dietary Fiber Content

This method is essentially the same as the method published by the AOAC (Official Method 991.43: Total, Soluble and Insoluble Dietary Fiber in Foods; Enzymatic-Gravimetric Method, MES-TRIS Buffer, First Action 1991, Final Action 1994). Here only isopropyl alcohol was used instead of ethanol.

### 5. Analysis of the Aroma-Active Compounds (VOC)

In order to be able to analyze low concentrations of VOCs in various samples using GC/MS, sample enrichment must be carried out prior to analysis. The needle trap technique (NT) was used to analyze the aroma-relevant ingredients of the various citrus fiber process samples. For the samples containing isopropyl (IPA), solid phase microextraction (SPME) was also used, as the high enrichment of the NT resulted in strong peak overlaps with IPA in the chromatogram up to retention times of approx. 13 min. With SPME, hardly any peak overlaps occurred due to the less strong enrichment. The respective samples were stored refrigerated until analysis.

### 5.1 Analysis of VOCs Using HS-SPME-GC/MS

The SPME combines sampling and enrichment of the analytes in one step and enables direct transfer of the analytes to a GC. For the SPM E method, a coated fiber is used in a special field sampler. The coated fiber is exposed to the sample for a defined time, wherein the analytes are adsorptively or absorptively bound depending on the coating material. After extraction, the analytes are thermally desorbed from the fiber coating in the GC injector.

### 5.1.1 Sample Preparation

For the analysis, a volume of approx. 5 mL, corresponding to 1-3 g of sample, was weighed into a 15 mL headspace vial. Prior to analysis, the sample was conditioned for 10 min at 40° C. with shaking. Immediately afterwards, the volatile substances were enriched by SPME from the headspace above the sample. The measurement conditions are as follows:
SPME phase: carboxen/polydimethylsiloxane (PDMS), 75 μm (Supelco, Sigma-Aldrich, Taufkirchen, Germany)
SPME enrichment duration: 30 min
Enrichment temperature: 40° C.
Desorption temperature: 290° C.
Desorption duration: 5 min

### 5.1.2 Description of the GC/MS System

The measurements were carried out on the following GC/MS system:

| | |
|---|---|
| Device | HP 6890N Gas Chromatograph (Agilent Technologies, |
| | Waldbronn) |
| Injector | split/splitless injector; splitless mode (45 s), |
| | pressure surge (30 s) |
| Separation | VF-624 ms, 30 m, I.D. 0.25 mm, film thickness 1.4 pm |
| column | |
| Carrier gas | Helium |
| Detector | HP 5973 Mass Selective Detector, scan mode: m/z 25-350 |
| Data | HP-ChemStation, version A.03.00 |
| acquisition | |

The individual substances were identified using mass spectra databases (Me. Lafferty, 2000; Wallace, 2020-Mass Spectra), retention indices (Wallace, 2020-Retention indices) and, if available, by comparing the retention times with those of standard substances.

McLafferty, F. (2000): Wley Registry of Mass Spectral Data, seventh ed, Wiley, New York.

Wallace, W. E. (2020): Mass Spectra/Retention Indices. NIST Chemistry WebBook, NIST Standard Reference Database Number 69. Eds. P. J. Linstrom and W. G. Mallard, National Institute of Standards and Technology, Gaithersburg MD, 20899, https://doi.org/10.18434/T4D303.

### 5.1.3 Determination of the Contents of the Sample Ingredients

The contents of the identified sample constituents were determined semi-quantitatively as toluene equivalents. For the calibration, three aliquots of an isopropanol sample were spiked with different toluene concentrations. The SPME enrichment and GC/MS analysis were then performed as described in sections 5.1.1 and 5.1.2. This resulted in a measuring range of 30 to 870 μg/L toluene. The calibration characteristics are shown in the table in section 5.2.3.

### 5.2 Analysis of VOCs Using Needle Trap GC/MS

The volatile components of the citrus fiber samples were identified by needle trap GC/MS and their concentrations were expressed as toluene equivalents.

In order to be able to analyze low concentrations of volatile organic compounds (VOC) in various samples using GC/MS, sample enrichment must be carried out prior to analysis. The needle trap technique was used to analyze the samples. The needle trap is a micro-adsorption unit consisting of a cannula (23 gauge) filled with a small amount (<1 mg) of strongly enriching sorbents. To enrich the volatile substances, a defined volume of air from the headspace above the sample is drawn through the needle trap using a pump, wherein the aroma-active substances are retained on the sorbent. The subsequent separation of the analytes from the sorbent material is carried out thermally in the GC injector.

### 5.2.1 Sample Enrichment:

For the analysis, a volume of approx. 5 mL, corresponding to 1.5-3 g of sample, was weighed into a 15 mL headspace vial. Prior to analysis, the sample was conditioned for 15 min at 40° C. with shaking. Immediately afterwards, the volatile substances were enriched from the headspace above the sample using the needle trap technique.

### Measuring Conditions:

| Needle Trap | Tenax TA/Carbopack X/Carboxes 1000 | |
|---|---|---|
| | (PAS Technology Germany, Magdala) | |
| Sample volume | 20 | mL |
| Flow rate | 3 | mL/min |
| Desorption temperature | 280° | C. |
| Desorption time | 45 | s |

The measurements were carried out on the GC/MS system described in section 5.1.2.

The individual substances were identified as described in section 5.1.3 using mass spectra databases, retention indices and, if available, by comparing the retention times with those of standard substances.

### 5.2.2 Contents of Aroma-Active Compounds:

The contents of the identified sample constituents were determined semi-quantitatively as toluene equivalents. For this purpose, it was assumed that the volatile substances were completely transferred from the sample into the headspace. For the calibration, 2.5 μL toluene was evaporated in a gas mouse. This standard gas was diluted to produce the various calibration gas concentrations. The enrichment and GC/MS analysis were then carried out as described in section 5.2.1. This resulted in a measuring range of 0.003 to 3.1 mg/m³ or 0.01 to 25 μg/kg in relation to the sample weight. The calibration characteristics are shown in the table under point 5.2.3.

### 5.2.3 Toluene Calibration Characteristics:

| | Measuring | Detection | | |
|---|---|---|---|---|
| | range | limit | | Method |
| | [μg/kg | [μg/kg | Correlation | variation |
| Method | or μl/kg] | or μl/kg] | coefficient | coefficient |
| SPME | 30-870 | 15 | 0.999 | 4.6% |
| NT | 0.01-25 | 0.5 | 0.9999 | 2.6% |

The method fulfills the requirements of DIN 32645 (2008) (Chemical analysis-Detection, detection and quantification limits under repeatability conditions-Definitions, procedures, evaluation. Beuth Verlag, Berlin) for the calibration parameters. The peak areas measured by GC/MS were used to determine the contents of the sample substances and the toluene equivalents were calculated using the toluene calibration data. The different response factors of the substances were not taken into account.

Result: Analyzed contents of aroma-active substances

For comparison purposes, a sample of “lemon peel fiber, unwashed” was analyzed under identical conditions. This sample was a flavedo rich lemon peel, which was dried at 60° C. and then ground to particles with a particle size of less than 500 μm. This sample was therefore not subjected to extraction using a water-based extraction medium and an alcohol-based extraction medium.

The analyzed contents of aroma-active substances are given in the following table for all samples tested. The unit in each case is “pg/kg toluene equivalents”.

| | Citrus fiber | Citrus fiber | Citrus |
|---|---|---|---|
| | according to the | according to the | peel, fiber, |
| | invention - lot A | invention - lot B | unwashed |
| Aldehydes | 3.20 | 3.30 | 794 |
| Carboxylic acids | 0.02 | 0.90 | 116 |
| Terpenes | 0.20 | 0.20 | 322520 |

In addition, the citrus fiber according to the invention was compared with other commercially available citrus fibers with regard to the content of aroma-active substances. The analyzed contents of selected aroma-active substances for the tested samples are given in the following table, wherein both the selected aroma-active compounds are shown, as well as the totals for the respective substance class. The unit in each case is “pg/kg toluene equivalents”, BG: limit of quantification (0.02 μg/kg toluene equivalents), n.n.: not detectable (<0.008 μg/kg toluene equivalents).

| | Citrus fiber | Ceam fiber | Citri-Fi 100 |
|---|---|---|---|
| | according to | Citrus fiber | Citrus fiber |
| | the invention | (Ceamsa) | (Fiberstar) |
| Aldehydes | | | |
| Acetaldehyde | 0.3 | 0.9 | 1.1 |
| Propanal | 0.08 | 0.3 | 4.3 |
| Butanal | <BG | 3.1 | 0.5 |
| (E)-2-Butenal | <BG | 0.6 | 0.09 |
| Pentanal | 0.1 | 25 | 1 |
| (E)-2-Pentanal | <BG | 0.4 | 0.04 |
| Hexanal | 1.1 | 131 | 13 |
| (E)-2-Hexanal | <BG | 0.6 | 0.3 |
| Heptanal | 0.06 | 8.7 | 0.5 |
| Sum aldehydes | 1.6 | 170.6 | 20.8 |
| Carboxylic acids | | | |
| formic acid | n.n. | 0.1 | 0.3 |
| acetic acid | n.n. | n.n. | 17 |
| propanoic acid | n.n. | 0.7 | 0.4 |
| valeric acid | <BG | 1.6 | <BG |
| caproic acid | <BG | 6.6 | 0.2 |
| Sum carboxylic acids | <BG | 9 | 17.9 |
| Terpenes | | | |
| alpha pinene | 0.06 | 0.7 | 0.1 |
| Limonene | 0.09 | 1 | 6.1 |
| p-cymol | <BG | 0.1 | >BG |
| Sum terpenes | 0.15 | 1.8 | 6.2 |

FIG. 2 also contains a table showing the contents of the aroma-active substances in the fiber material after the respective extraction steps and thus illustrates the course of the extraction and the gradual depletion of the flavorings. This clearly shows that even the aqueous extraction steps lead to a significant reduction in the aroma-active substances. It is worth noting, however, that the content of carboxylic acids initially increases during aqueous extraction, which is due to the fact that the difficult-to-extract carboxylates present in the starting material are profanated by the acidic digestion or the acidic stopping of the enzymatic digestion in step (b) and can only then be easily extracted from the material. The unit in each case is “pg/kg toluene equivalents”.

### EVALUATION

The method described here can be used to reproducibly produce functionalized flavoring-depleted citrus fibers. Compared to fibers which have not been subjected to an extraction described here, in particular using a water-based extraction medium and an alcohol-based extraction medium, these fibers have a significantly lower intrinsic aroma with satisfactory functional properties. Consequently, the functionalized citrus fibers described here are particularly versatile.

### LIST OF REFERENCE CHARACTERS

10 citrus pomace
20 digestion
30a 1. solid-liquid separation decanter
30b 2. solid-liquid separation separator
35 extraction using an aqueous extraction medium and subsequent wet sieving
40 solid-liquid separation decanter
50 1. extraction with alcohol
60 solid-liquid separation decanter
70 2. extraction with alcohol
80 solid-liquid separation decanter
90 3. extraction with alcohol
100 solid-liquid separation with screw press with TS≥45% by weight
110 vacuum drying
120 obtained functionalized and sensory neutral citrus fiber

1-21. (canceled)

22. A method for the production of a flavoring-depleted, functionalized citrus fiber, the method comprising steps of:
(a) providing an aqueous suspension of a raw material containing cell wall material of an edible citrus fruit;
(b) digesting the raw material in the aqueous suspension by at least one of
i. incubation of the aqueous suspension of the raw material at an acidic pH value,
ii. enzymatic treatment of the aqueous suspension of the raw material, and
iii. introduction of mechanical energy into the aqueous suspension of the raw material
to obtain a digested material;
(c) separating a first fraction of flavorings from the digested material of step (b) via single or multi-stage separation to obtain a digested and flavoring-depleted material;
(d) optionally extracting a second fraction of flavorings from the digested and flavoring-depleted material of step (c) with a water-based extraction medium at a temperature of 30° C. to 90° C.;
(e) optionally separating the digested and flavoring-depleted material of step (d) from the water-based extraction medium;
(f) extracting a third fraction of flavorings by contacting the digested and flavoring-depleted material from step (c) or step (e) with an alcohol-based extraction medium;
(g) separating the digested and flavoring-depleted material from step (f) from the alcohol-based extraction medium;
(h) drying the digested and flavoring-depleted material from step (g) to obtain the flavoring-depleted, functionalized citrus fiber,
wherein step (g) is carried out such that the obtained flavoring-depleted, functionalized citrus fiber has a dry matter content of ≥30% by weight.

23. The method according to claim 22, wherein the raw material is a residue from the processing of citrus fruits.

24. The method according to claim 22, wherein the raw material is selected from the group consisting of citrus peel, citrus albedo, citrus flavedo, citrus vesicle, citrus membrane, citrus pomace, and citrus pulp.

25. The method according to claim 22, wherein digestion of the raw material in the aqueous suspension in step (b) is performed by the introduction of mechanical energy into the aqueous suspension of the raw material in step (b), and wherein the introduction of mechanical energy into the aqueous suspension of the raw material in step (b) is carried out using a method selected from the group consisting of high shear treatment, pressure homogenization, colloidal grinding, extrusion, ultrasonic treatment, microfluidizer, spider disperser and combinations thereof.

26. The method according to claim 22, wherein digestion of the raw material in the aqueous suspension in step (b) is performed by the incubation at the acidic pH value, and wherein the incubation in step (b) satisfies one or more of the following conditions:
i. an organic acid is used;
ii. a mineral acid is used;
iii. the acidic pH value is between pH=0.5 and pH=4.0;
iv. the incubation is carried out at a temperature between 60° C. and 95° C.;
v. the incubation is carried out over a period of 60 min to 8 hours;
vi. the suspension has a dry mass of between 0.5% and 5% by weight; and
vii. the suspension is stirred and/or shaken during the incubation.

27. The method according to claim 22, wherein digestion of the raw material in the aqueous suspension in step (b) is performed by the enzymatic treatment, and wherein the enzymatic treatment is carried out with one or more of the following enzymes: pectinases, cellulases, and hemicellulases.

28. The method according to claim 22, wherein the separation in step (c) is carried out with a decanter, a separator and/or a belt press.

29. The method according to claim 22, wherein extraction of the second fraction of flavorings is performed in step (d), and wherein the extraction of the second fraction fulfils one or more of the following conditions:
i. the water-based extraction medium is distilled water or drinking water;
ii. the water-based extraction medium is a salt solution with an ionic strength of I<0.2 mol/1;
iii. the water-based extraction medium has a pH value of 1 to 7;
iv. the extraction takes place at a temperature between 30° C. and 90° C.;
V. contact with the water-based extraction medium takes place over a period of between 10 min and 2 h;
vi. the dry mass in the aqueous extraction mixture consisting of the water-based extraction medium and the material separated in step (c) is between 0.1% by weight and 5% by weight;
vii. the extraction step is carried out in a batch method; and
viii. the extraction step is carried out with stirring and/or shaking.

30. The method according to claim 22, wherein the separation of the digested and flavoring-depleted material from the water-based extraction medium according to step (e) is carried out by means of a decanter, a belt press and/or a separator.

31. The method according to claim 22, wherein extraction according to step (d) with subsequent separation according to step (e) is repeated several times.

32. The method according to claim 22, wherein the extraction in step (f) satisfies one or more of the following conditions:
i. the alcohol is selected from the group consisting of methanol, ethanol and isopropanol;
ii. the alcohol-based extraction medium has an alcohol content of between 50 and 95% by volume;
iii. the extraction takes place at a temperature between 40° C. and 75° C.;
iv. the contacting with the alcohol-based extraction medium takes place over a period of between 60 min and 10 h;
v. the dry mass in the alcoholic extraction mixture consisting of the alcohol-based extraction medium and the material separated in step (d) is between 0.5% and 15% by weight;
vi. the extraction is carried out using a countercurrent process; and
vii. the extraction step is carried out with stirring and/or shaking.

33. The method according to claim 22, wherein the separation according to step (g) is carried out by means of a screw press, a decanter, a belt press and/or a separator.

34. The method according to claim 22, wherein the extraction according to step (f) with subsequent separation according to step (g) is repeated several times and at least in the last of these several times the separation in step (g) is carried out in such a way that the separated material has a dry substance content of ≥30% by weight.

35. The method according to claim 22, wherein the method further comprises, after drying in step (h), a comminution, grinding and/or sieving step to obtain particles.

36. A flavoring-depleted, functionalized citrus fiber having a total content of aldehydes of less than 500 μg/kg toluene equivalents obtained by the method according to claim 22.

37. A flavoring-depleted, functionalized citrus fiber having a total content of carboxylic acids of less than 500 μg/kg of toluene equivalents obtained by the method according to claim 22.

38. A flavoring-depleted, functionalized citrus fiber having a total terpene content of less than 1000 μg/kg toluene equivalents obtained by the method according to claim 22.

39. A flavoring-depleted, functionalized citrus fiber having a water-binding capacity of more than 5 g/g.

40. A mixture comprising a flavoring-depleted, functionalized citrus fiber according to claim 36, and an isolated pectin.

41. A food product, feed product, food supplement, beverage, cosmetic product, pharmaceutical product, detergent product, cleaning product or a medical device manufactured using the flavoring-depleted, functionalized citrus fiber according to claim 36.
